(19) United States
(12) Patent Application Publication
(10) Pub. No.: US 2023/0288938 A1
(43) Pub. Date: Sep. 14, 2023

(54) AUTOMATED ROBOTIC DISINFECTION SYSTEM AND METHOD

(71) Applicant: Haystack Robotics, Inc., Portland, OR (US)

(72) Inventors: Paul R. West, Portland, OR (US); Mahadev Eakambaram, Portland, OR (US)

(21) Appl. No.: 18/121,538

(22) Filed: Mar. 14, 2023

Related U.S. Application Data
(60) Provisional application No. 63/319,707, filed on Mar. 14, 2022.

(51) Int. Cl. G05D 1/02; G05B 19/4155; G05D 1/00; A61L 2/24; A61L 2/10

(52) U.S. Cl. CPC G05D 1/0219; G05B 19/4155; G05D 1/0094; A61L 2/24; A61L 2/10; G05B 2219/50391; G05D 2201/0203; A61L 2202/16; A61L 2202/11; A61L 2202/14; A61L 2202/25

(57) ABSTRACT

Systems and methods are described for disinfecting a space, such as an interior room. In some aspects, at least one object in the space may be identified, such as by a mobile disinfecting device (device) including an ultraviolet lamp. Next, the object(s) may be categorizing as a certain type based on whether the object allows some light to pass through or around it. A route for the device may be determined, where the route includes a path and speed of the device corresponding to different segments of the path to yield a dosage score (ultraviolet exposure) that meets or exceeds a threshold value for different areas of the space. In some cases, determining the route further includes determining at least one of a speed, a modified path, or an ultraviolet radiation characteristic for at least one segment of the path based on the categorization of the object(s).

### CROSS-REFERENCE TO RELATED APPLICATIONS

This application claims the benefit of U.S. Provisional Patent Application No. 63/319,707, filed Mar. 14, 2022, the disclosure of which is incorporated herein by reference in its entirety.

### BACKGROUND

In various contexts, disinfection, such as may be used in hospitals, hotel rooms, common areas in various buildings, etc., is becoming increasingly automated. For example, robots and other mechanical devices can be used and coupled with ultraviolet (UV) lamps to expose areas around a room to disinfect for viruses and bacteria. While there are many advantages to this automated approach (safety, time/money savings), there are also challenges that arise due to the robot/device occasionally having limited access to an environment. For example, typically such devices can only move along the floor, and do not have proximity access to locations such as the center of a bed or a large table or other large objects. Accordingly, a need exists for improved techniques for disinfecting spaces, primarily interior spaces.

### BRIEF DESCRIPTION OF THE DRAWINGS

Various techniques will be described with reference to the drawings, in which:

FIG. 1 illustrates an example diagram of UV exposure dosage as a function of distance from a UV lamp, according to at least one embodiment;

FIG. 2 illustrates an example diagram of different classification of objects based on whether light can pass through or around the objects, according to at least one embodiment;

FIG. 3 illustrates an example of a representation of accumulated UV dosage score relative to an overhead view of a room, according to at least one embodiment;

FIG. 4 illustrates an example schematic of a mobile UV exposure/disinfecting device including hardware components, according to at least one embodiment;

FIG. 5 illustrates another example schematic of a mobile UV exposure/disinfecting device including at least one mirror device, according to at least one embodiment;

FIGS. 6-9 illustrate example UV exposure maps of the mobile UV exposure/disinfecting device of FIG. 5 with the mirror device in different configurations, according to at least one embodiment;

FIG. 10 illustrates another example of a representation of accumulated UV dosage score relative to an overhead view of a room, according to at least one embodiment;

FIG. 11 illustrates an example of a representation of accumulated UV dosage score relative to an overhead view of a room correlated to dwell time/speed of a mobile UV exposure/disinfecting device, according to at least one embodiment;

FIG. 12 illustrates an overhead view of a room map with specific objectives identified for UV exposure, according to at least one embodiment; and

FIGS. 13 and 14 illustrate example processes for disinfecting a room, according to at least one embodiment.

### DETAILED DESCRIPTION

Various embodiments discussed herein relate to disinfecting areas, such as indoor areas, by maximizing or optimizing time efficiency of a given mobile UV lamp devices' path, lamp power, movement speed, and or lamp direction or redirection. The center of large objects (e.g., beds, chairs, desks, etc.) may not be fully disinfected with a default disinfection time, given the distance the center of these large objects are from a UV lamp, such as may be attached to a mobile unit or device. Accordingly, various embodiments discussed herein include systems and methods configured to identify objects that require longer disinfection times (e.g., based on the size of the object) and to implement disinfection decisions, including movement decisions, lamp power decisions, and/or lamp direction decisions, in a physical room, to ultimately decrease the time taken to disinfect a room to a given disinfection threshold.

In some examples, UV devices or disinfecting devices, such as referred to herein generally as UV robots or mobile disinfecting devices, can globally decrease their speed to ensure the disinfection radius encompasses the largest or larger objects in the room. However, in various embodiments, globally slowing the robot movement would result is very long disinfection cycles, as the robot would not move quickly in areas without large obstacles. In view of the foregoing, a need exists for an improved UV disinfection system and method for disinfecting indoor spaces in an effort to overcome the aforementioned obstacles and deficiencies of conventional automated cleaning and disinfection systems.

Ultraviolet (UV) disinfection destroys viruses and bacteria as a function of light energy, distance from lamp to object, and exposure time for the object. In a situation where a room has large objects outside the disinfection radius of the lamp (e.g., a bed, large table, etc.), a viable way to disinfect the area with a given robot can be to increase the dwell time (e.g., slow robot movement) to disinfect the area, such that the UV light is exposed to the center or far side of the large objects for a longer duration. The described techniques enable a robot or other device capable of movement at least along a substantially horizontal plane, to automatically slow movement to increase the disinfection radius around the large objects (increased disinfection), and increase movement speed around when no large obstacles are present.

Various embodiments discussed herein include robots that can be configured to disinfect indoor spaces faster, using less energy, and less battery power. Various embodiments discussed herein include robots that can be configured to ensure that the room is fully disinfected (or disinfected to a desired disinfection threshold), as some UV disinfection robots may not kill viruses in the center of beds, tables, or other large objects. In various aspects, the described devices, systems and techniques may provide for a number of benefits and advantages, such as a more efficient way to disinfect a given area, including reduced time to disinfect an area when compared to a fixed speed or fixed radiation disinfecting device, more efficient energy usage of the disinfecting device via being able to module UV lamp intensity, direction, and path of the device etc., such that a more consistent dosage score may be delivered to an entire area without delivering excessive UV radiation to certain areas, and various other advantages, as will be described in greater detail below. In some examples, the described techniques may be capable of disinfecting a fifteen by fifteen foot room in approximately ten minutes compared to a fixed speed/fixed radiation device, which may take up to twenty five minutes to provide the same level of disinfection to the same area.

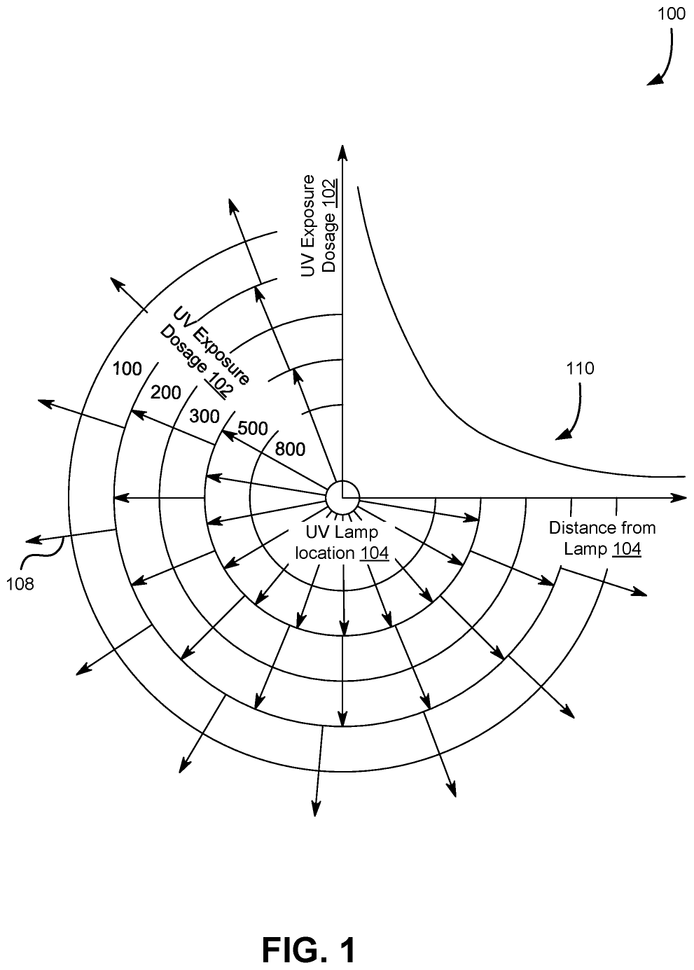

UV disinfection is used to kill viruses and bacteria. In order to do so, a radiation dosage is needed to destroy the DNA of these germs. The dosage of the UV lamps tapers off as a function of distance from the lamp, proportional to 1/a² where a is the distance from the lamp. FIG. 1 illustrates an example diagram 100 of UV exposure dosage 102 as a function of distance 104 from a UV lamp 106 (including graph 110), where the amount of the dosage is represented by the density/number of arrows 108. As illustrated in FIG. 1, distances close to the robot's lamps will receive a larger UV dosage (e.g., more arrows located in the closest circle relative to UV lamp location 104), and kill more viruses/bacteria in a shorter exposure time to the UV lamp 104, while objects at distances further from the robot's lamp or lamps 104 will receive less UV dosage and kill less viruses/bacteria (e.g., less arrows in the outer most circle away from UV lamp location 104). In various embodiments, at UV lamp locations, the surrounding area can receive a “dosage score” as a function of its distance from the lamp(s). The areas close to the lamp can receive a large dose (e.g., will receive a high dosage score) and the area away from the lamps receives a low dose (e.g., low dose score), in the same exposure time. Using the techniques described herein, the amount of time a given location or area is exposed to UV lamp radiation can be modified, such as by adjusting the speed/dwell time of a mobile UV lamp unit or autonomous vehicle equipped with at least one UV lamp. In yet some instances, as will be described in greater detail below, intensity of the UV lamp may also be modified to increase the UV exposure of certain areas, either by increasing the output of one or more UV lamps, activating additional UV lamps, changing the exposure direction of the one or more lamps, or a combination thereof.

In some aspects, to aid in the process of determining a route for a mobile UV unit to traverse, such as in an indoor space or room, to provide a threshold level of disinfection to the entire or substantially all of the indoor space, objects in the space may be categorized. The categorization may be used, in turn to determine a speed (or alternatively a dwell time, such as if the unit comes to stop) of the mobile UV lamp unit at various locations within the space to be disinfected. In accordance with various embodiments, objects in an indoor space can be separated into a plurality of categories, with some examples including two or more categories. In some examples, distinction between different types of objects can be determined by sensor inputs into the system, including lidar, cameras, and the like, which can determine the location, size, and/or shape of the obstacles.

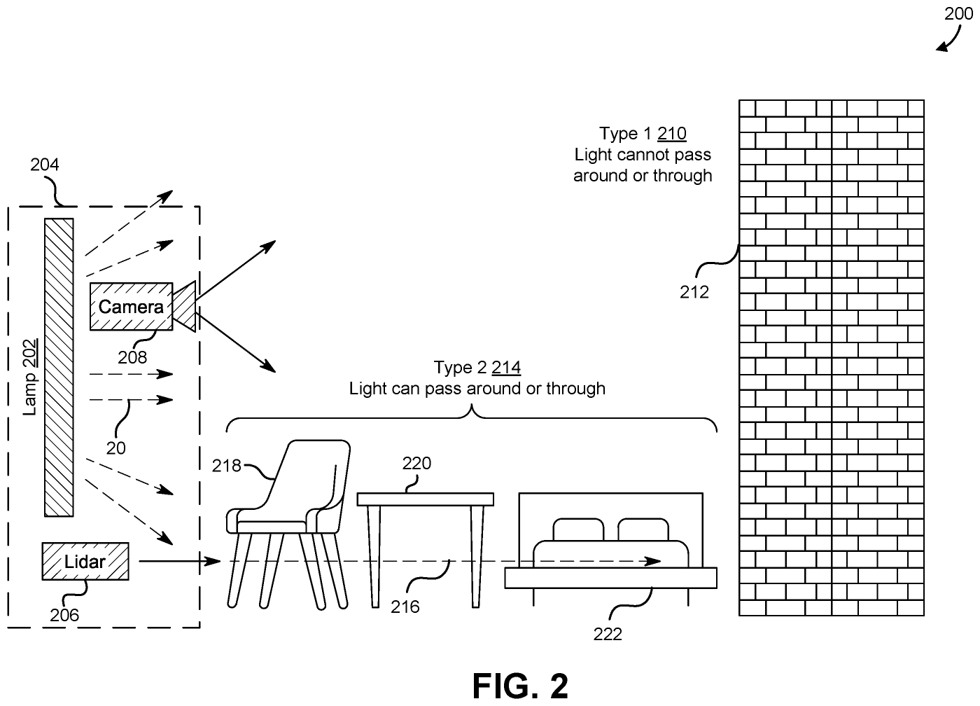

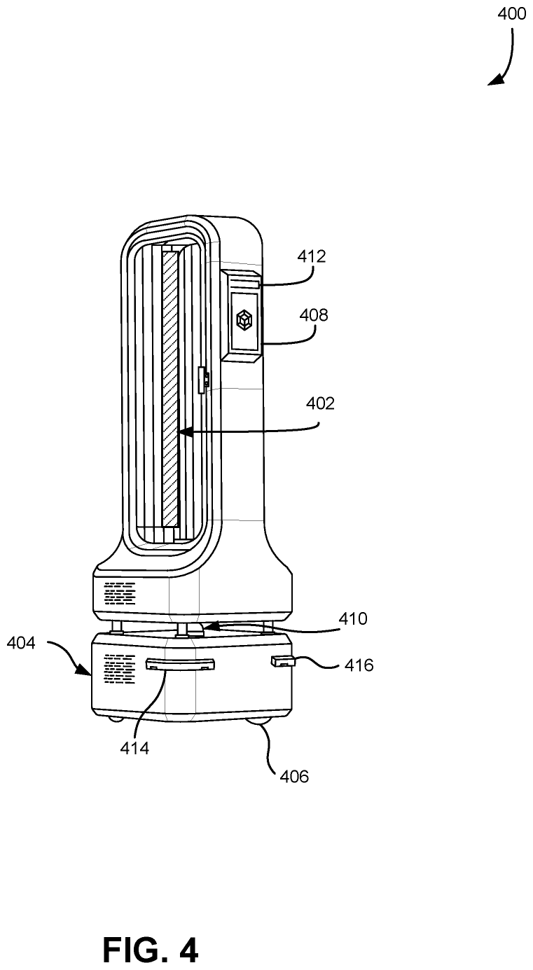

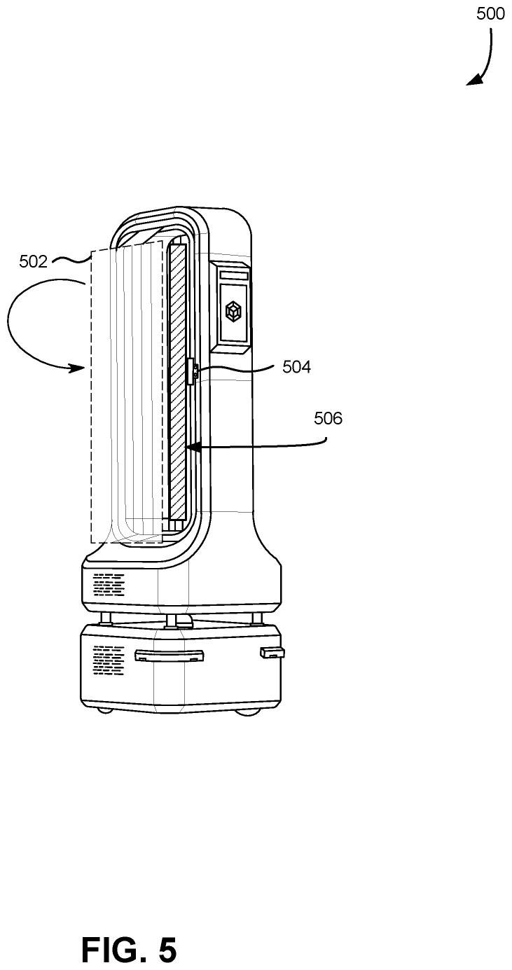

FIG. 2 illustrates an example diagram 200 of different classifications of objects based on whether light can pass through or around the objects. As illustrated in FIG. 2, one or more UV lamps 202 may be associated with a mobile disinfecting device or robot 204 (also referred to herein as a mobile UV device, or autonomous disinfecting device). In some cases, the UV device 204 may also include one or more sensors, such as one or more lidar devices 206, camera devices 208, and various other sensor, such as may be used to sense objects proximate to the UV device 204. The sensors 206, 208 may be utilized to determine what type or category objects, such as may be found in an indoor area or area to be disinfected, should be classified as. More details about an example autonomous vehicle equipped with at least one UV lamp will be described in greater detail below in reference to FIGS. 4 and 5.

In some examples, two categories may be used with a first type or type 1 category 210 including obstacles such as walls 212 or doors (not shown) that extend above and below the height of the UV lamps 202 and do not allow light to pass through or around the object. A second type or type 2 category 214 can include objects that light can pass through or around (indicated by dotted arrow 216), such as chairs 218, tables 220, beds 222, etc. In some examples, the distinction between Type 1 and Type 2 objects can be determined by sensor inputs into the system, including lidar 206, cameras 208, and the like, which can determine the location, size, and/or shape of the obstacles, such as illustrated in diagram 200 of FIG. 2. In some aspects, using a camera device a, lidar, sonar, and/or proximity sensors, a point cloud may be generated of the room to identify objects in the room, and in turn those sensors may be used to determine whether light can pass through or around the objects, to classify or categorize the objects accordingly. In turn, the type of object may then be used to determine if the object needs to be exposed to UV lamp radiation for a shorter or longer duration of time. In some examples, type 1 objects may need less of an exposure dosage (e.g., measured in UV lamp intensity multiplied by time) than type 2 objects. This may be accomplished via the UV device traveling at a greater rate of speed near type 1 objects, traveling a greater distance away from type 1 objects, supplying less power to the one or more UV lamps when proximate to the type 1 object, only activating a subset or less UV lamps proximate to the type 1 object when the device has a plurality of UV lamps, and vis versa (e.g., more time, less speed for type 2 objects). In yet some cases, as will be descried in greater detail below, mirror or other reflective devices may be used to change direction and/or intensity of UV lamp exposure to areas proximate to the UV device.

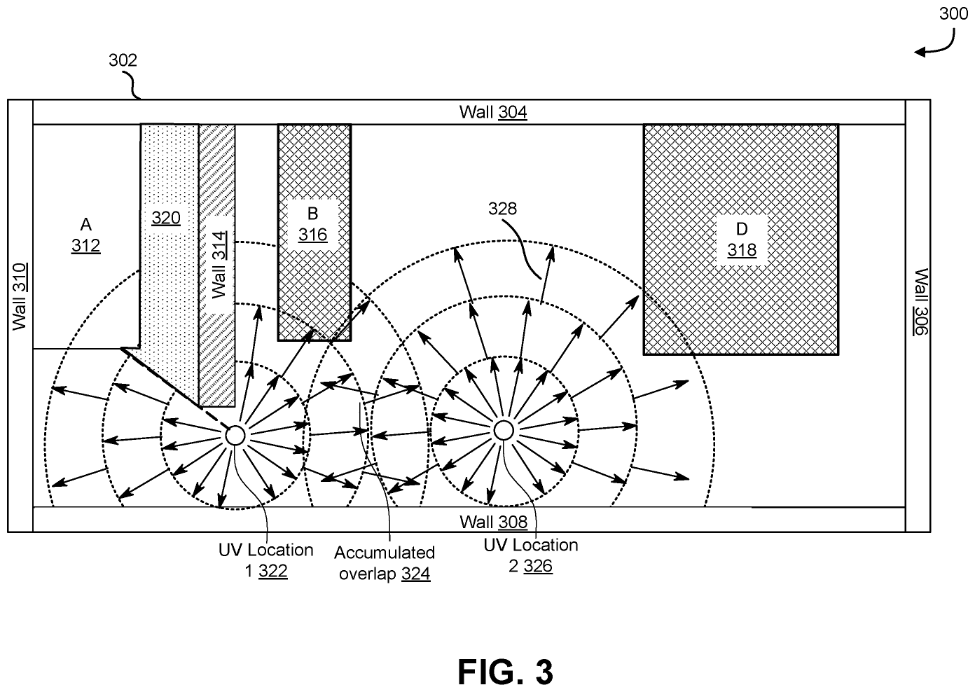

FIG. 3 illustrates an example diagram 300 of a representation of accumulated UV dosage score, indicated by density of arrows 328, relative to an overhead view of a room 302, defined by walls 304, 306, 308, and 310. From the example diagram 300, it can be seen that objects such as object B 316 and object D 318 (e.g., type 2 object) can receive a normal dosage score, while the wall object 314 (e.g., type 1 object) blocks light in this example and creates a shadow region 320 which in this example will not receive any UV light, and a no (zero) dosage score from the UV lamp location 322. As illustrated in FIG. 3, the “dosage score” is illustrated as density of arrows 328 in different areas (illustrated as circles) around UV lamp location 1 322, and areas around UV lamp location 2 326. For obstacles or objects (such as walls or other type 1 objects) that block the UV light, the area in the shadow region 320 will not receive any dosage score in various examples. In some cases, there may be an accumulated overlap area 324 near the lamps 322, 326 in this example at different times where the UV dosage is accumulated with time.

In various embodiments, with time, the robot and UV lamps move—covering the obstacle-free areas of the room. At each point in time (or various suitable points in time), the location and corresponding dosage scores can be applied to the location in the room. The dosage score for each point in time can add cumulatively in various examples, such that areas closer to the UV light for more time, or multiple passes will receive more UV dosage and higher dosage scores. Areas further from the UV location, for less time, and fewer passes will receive less UV dosage and corresponding score in various examples.

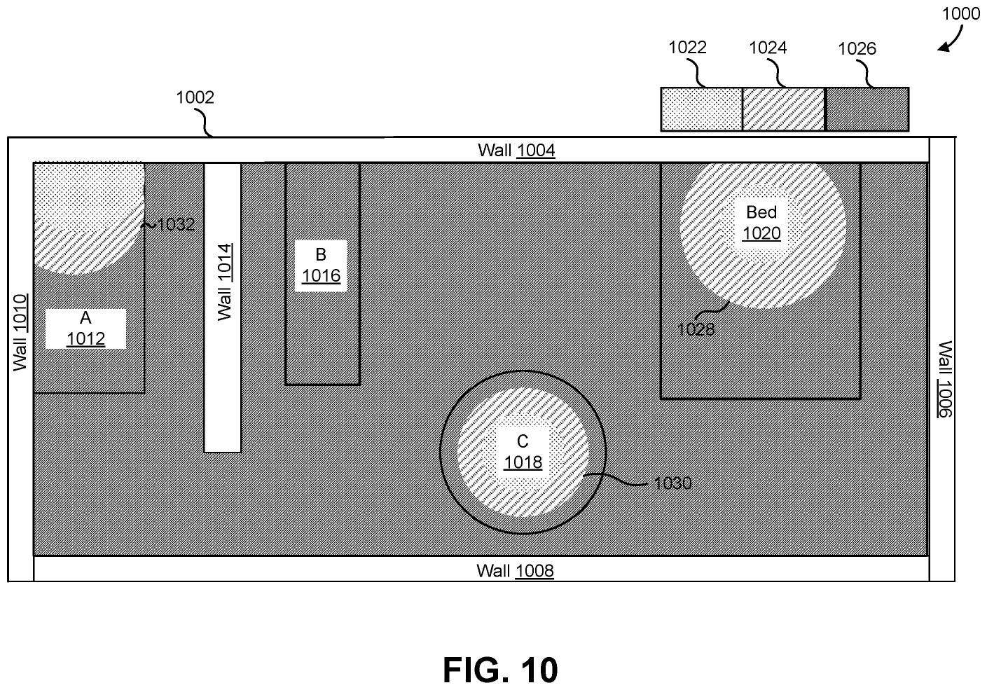

In various examples, the UV dosage score may be the total UV lamp radiation an area has been exposed to over an entire route of a mobile UV device, as described herein. In some cases, the mobile disinfecting device (or backend computing system in communication with the mobile UV device) may record the actual route through a room to be disinfected, including locations, speeds, dwell time, UV lamp number and orientation or direction of the resulting UV radiation, UV lamp intensity, and/or any mirror operation or position. Using some or all of this information, a dosage score for the space to be disinfected may be determined. The dosage score may be represented by a heat map, such as will be described in greater detail below in reference to FIGS. 10-12.

In some embodiments, a global threshold dosage score can be set for the room that is required for killing a given virus or bacteria to a given disinfection level. For example, some virus/bacteria need a higher UV dosage to destroy them, therefore requiring a higher dosage threshold score. (e.g., MRSA and SARS-COV-2 may require different amounts of UV dosage). Some types of rooms need higher disinfection rates. For example, a hospital operation room may need to be 99.9999% disinfected, while a hotel room may only require 99.99% disinfection. Therefore, the hospital may need a higher threshold dosage score in various examples, than other types of rooms or spaces.

In some embodiments, in addition to or as an alternative to a global threshold dosage score, an object-of-interest dosage score can be set for specific areas or locations in the room. This can be detected using object inference artificial intelligence (AI) techniques, or other suitable method. For example, this could be used for: (a) high-touch objects: such as door handles, light switches, faucet handles, etc., (b) highly sensitive objects with a high probability of infection: such as toothbrush, surgical or dental equipment, and/or (c) other objects of particular importance to various entities, situations, etc., which may be customizable for different applications.

In various embodiments, as the UV robot traverses the indoor room, the dosage score is accumulated. For areas of the room that are below the threshold dosage score, the robot can do one or more of the following to increase and reach the required dosage. In a first example, the UV robot can move closer to the area with insufficient dosage score. Moving closer to the location can increase the radiation intensity and can shorten the time required to disinfect the area. In some examples, such a method can be a primary technique used as the robot moves throughout the room. In another example, some areas of a room (such as the center of a large bed for example) may have insufficient dosage score after the robot covers the obstacle-free areas of the room. These areas can get sufficient dosage by a) bringing the robot as close as possible to the low-score area, and/or b) increasing the time the robot stays/dwells in the area (e.g., increase the dwell time of the UV robot) ad/or c) increase the intensity of the one or more UV lamps, as will be described in greater detail below. Because the dosage score is cumulative with time, the longer the robot stays in a location, the surrounding area's dosage score can continue to increase until the required dosage score is reached.

FIG. 4 illustrates an example schematic of a UV exposure device or mobile disinfecting device 400 including hardware components. In some examples, one or more UV lamps 402 may be attached to an autonomous vehicle unit or base 404, such that may navigate various spaces, such as the floor of a room. The UV lamps 402 may be supplied with power (not illustrated) to activate the one or more lamps 402 at a given intensity to disinfect an area proximate to the one or more lamps.

In some cases, the one or more UV lamps 402 may include one or more disinfecting lights (e.g., UV light) or other suitable disinfecting elements. In some cases, the one or more UV lamps may include one or more germicidal lamps with wavelengths in the range 200 nm to 280 nm (*such as at wavelengths of 254 nm, which are commonly used for this purpose) that can kill germs, bacteria, pathogens and/or viruses. The one or more UV lamps 404 may provide sterilization, also known as UV disinfection or ultraviolet germicidal irradiation (UVGI), which may break down certain chemical bonds and scramble the structure of DNA, RNA and proteins, causing a microorganism to be unable to multiply. In some cases, the one or more UV lamps 402 may have a fixed placement and/or fixed directionally on the mobile disinfecting device, such as producing a radiation pattern that spans part way around to all the way around (e.g., anywhere from 15, 30, 45, 90, degrees, all the way to 360 degrees or any amount in between) the mobile device. In some cases, the UV lamp(s) 402 may be placed a certain height above the bottom of the base unit 404, such as to be able to project to the floor or ground surface upon which the device 400 may travel. In some examples, this height may be 6, 12, 15, 20 inches, or various other distances, such as, in some cases, may be selected based on the application (e.g., size of space to be disinfected). In some aspects, the UV lamp(s) may be any of a variety of dimensions, lengths, including 4, 5, 6, or 7 feet tall, such as may be selected based on applicant (e.g., height of ceiling/walls of space to be disinfected).

In various examples, the mobile disinfecting device 400 (also referred to herein as a robot or autonomous vehicle), may include various components to enable motion, steering, speed control, etc., and various other motion controls, such as may form base unit or autonomous vehicle unit 404. Various definitions of robot or autonomous vehicle can include some type of propulsion device such as a motor which may be powered by a power source, including any of a variety of battery technology, run on various types of fuels, including gasoline diesel, propane, and various other types of fuels depending on application. In some cases, the robot or autonomous vehicle may also include any of a number of wheels 406 (e.g., 1, 2, 3, 4, 5 etc.) made of various materials, a chassis or frame (made of any of a variety of materials including plastics, composites, metals, etc.), a battery or other power source, and/or one or more implementations of a computing device 408.

In some cases, the computing device 408 may include one or more memory devices, one or more processors or CPUs, various network connectivity devices, such as Wi-Fi, Bluetooth, etc., and various other components to facilitate the mobile disinfecting device 400 to execute route planning, route execution, operate one or more UV lamp devices, operate one or more sensor devices, and communicate with one or more back-end services to provide reports and/or remote control of the mobile disinfecting device 400. In some cases, the computing device or devices may be placed on one or more logic boards, chips, etc. In some implementations, the computing device 408 may include one or more of a CPU, GPU, memory, wireless internet, Bluetooth, inputs (e.g., USB), etc. In some cases, the memory of the computing device 408 may include a computer readable medium that stores instructions (e.g., software) that when executed configures the robot to perform one or more of the functionalities, methods, or the like, described herein.

In some aspects, mobile disinfecting device 400 may include various sensors, in communication with computing device 408. In some cases, the one or more sensors may include one or more of the following: one or more lidar sensors 410, one or more cameras 412, one or more proximity sensors 414, one or more cliff sensors 416, one or more sonar sensors (not illustrated), and various other sensors that may be used to identify objects proximate to mobile disinfecting device 400 and/or determine and safely execute a route for mobile disinfecting device 400 to travel. In some cases, the output of the one or more sensors can be used as inputs to build a map of the room or space and detect the presence and/or distance of other objects and obstacles. These obstacles in various examples can include the presence of static objects (e.g., walls, chairs, tables), dynamic objects (people, animals, other robots, etc.), or the absence of a physical object (e.g., a step or other hole in the ground). In some cases, the detection of one or more moving objects (e.g., people, animals, other machinery, etc.), may trigger stopping motion of the mobile disinfecting unit 400 and/or turning-off the one or more UV lamps 402/redirecting the UV radiation away from the moving object. In some cases, the one or more camera devices 412 may include one or more digital video cameras (e.g., 2D/3D) which can be used to capture images of the physical space around the robot or for other suitable purposes.

In some cases, in order to increase the disinfection radiation in a desired direction, along with increasing the dwell time in a given area, mirrors can also be used to steer more light in a desired direction. FIG. 5 illustrates another example schematic of a mobile UV exposure device 500 including at least one mirror device 502. As illustrated in FIG. 5, a mirror's 502 position can be rotated around the a mobile UV exposure device 500 using a motor. In various examples, mobile UV exposure device 500 may include one or more aspects of mobile disinfecting device 400 described above in reference to FIG. 4; and for the sake of brevity, those similar aspects will not be described again here.

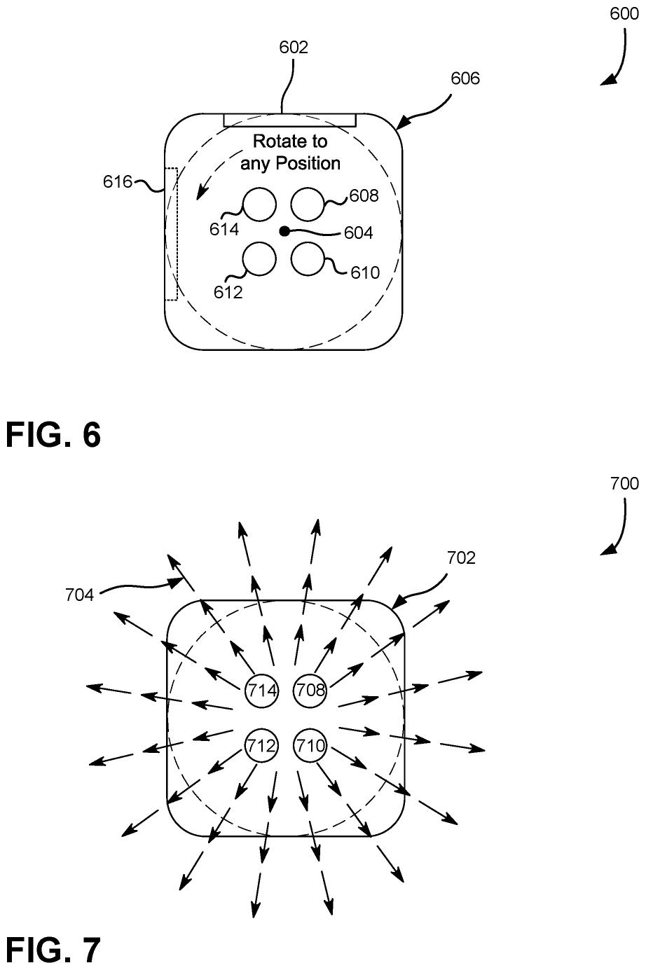

In some examples, mobile UV exposure device 500 may include one or more mechanical devices 504 for placing one or more mirrors 502 (or other surface that at least partially reflects UV light/radiation) in a radiation direction of one or more UV lamps or lights 506. In some cases, the mirror system may include one or more motors 504 that move a mirror surface in front of one or more UV lamps 506. In some cases, the mirror system may include at least one or more pivots such that upon application of force from a motor, the mirror surface can rotate about the one or more pivot points, to align the mirror surface in a direction of radiation of the one or more UV lamps 506. In one example, a planar structure including a mirror surface may be mounted about a vertical rod (or pivots at the top and bottom of the mirrored surface), proximate to the UV lamp, such that upon rotation, the mirror surface may block reflect light in one or more desired directions. Example mirror device configurations will be described in greater detail below in reference to FIGS. 6-9.

In some examples, a mirror system 504 may include one or more arms or other mechanical devices that can move in one or multiple directions, such as may be moved by motors, hydraulic systems, and so on, to provide greater flexibility in modification of the radiation pattern/to change the radiation pattern. In yet some cases, one or more UV lamps 506 may be movably secured to the device 500, such that the location of the UV lamp itself can be changed, such as to more quickly provide a high UV dosage score to an area farther away from the device 500. For example, the device 500 may include a robotic arm with a UV lamp attached to it. The arm may be moved when the device 500 identifies a large object that needs disinfecting (e.g., a type 1 object). The arm of the device may then be moved such that the attached UV lamp(s) is closer to the object, such as to disinfect a center of the object more quickly. In some cases, this may be more time efficient than having the device 500 move to the other side of the object and increase its dwell time to ensure that the center or far side of the identified objects receives the requisite UV exposure.

In some aspects, one or more operations or processes involved in generating the route for the mobile disinfecting device 400 and/or 500 may be performed locally via the computing device or system 408 of the device 400/500. In yet other aspects, one or more operations or processes involved in generating the route may be performed by remote computing resources, such as may communicate with the mobile disinfecting device 400 and/or 500 via one or more networks. In some aspects, the remote computing resources may include servers and/or other hardware devise, cloud computing resources, such as virtual machine and/or software containers/isolated execution environments, provided by a disinfecting service or third party provider, such as a computing resource service provider (e.g., Amazon Web Services, Microsoft Azure, etc.). In some cases, the resource intensive tasks may be performed by the remote computing resources to converse power and/or reduce complexity of the mobile disinfecting device 400 and/or 500.

FIGS. 6-9 illustrate example UV exposure maps 600, 700, 800, 900 of the UV exposure device of FIG. 5 with one or more mirror devices in different configurations. FIG. 6 illustrates shows a top-down view 600 of an example of how a mirror or other reflective surface 602 is rotated around the central axis 604 of the mobile disinfecting device 606, around the UV lamp bulbs 608, 610, 612, 614. In some aspects, the mirror surface or structure 602 may be rotated any amount (e.g., any number of degrees) about the central axis 604, As illustrated, mirror 602 may be rotated 90 degrees counter clockwise to position 616 from the original position 602.

FIG. 7 illustrates a typical UV radiation pattern of lamps 700 of a mobile disinfecting device 702 (without any mirrors), represented by arrows 704 extending outward from lamps 708, 710, 712, 714 in an even distribution in all directions. It should be appreciated, that in some implementations, there may be one or more structures of the mobile disinfecting device 702, such as support pillars and such, that may block out small portions of the radiation pattern, depending on the number of UV lamps equipped on the mobile UV device.

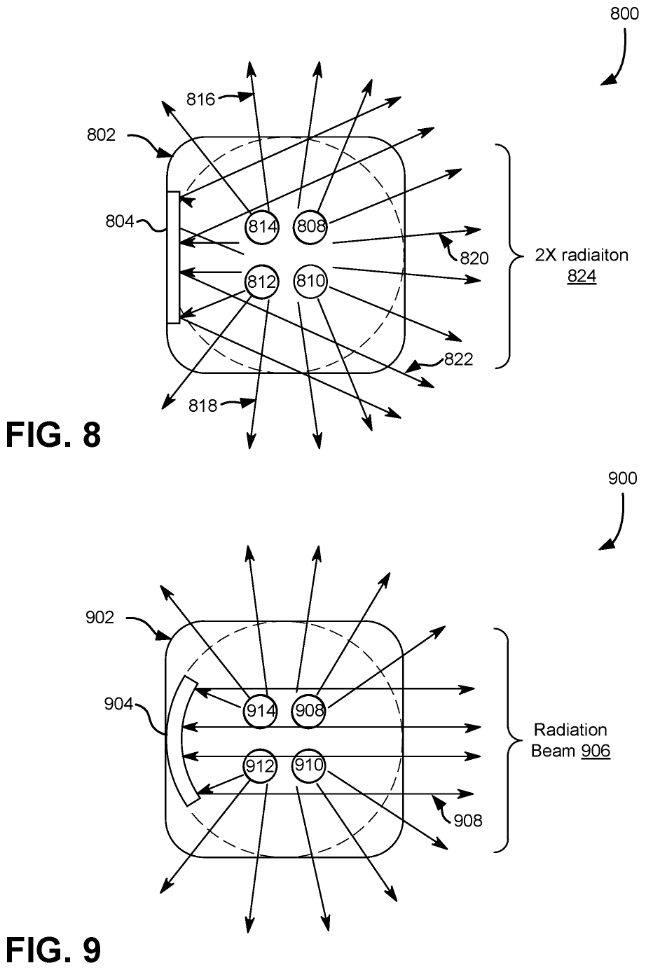

FIG. 8 illustrates an example radiation pattern 800 of a mobile disinfecting device 802 with a mirror surface 804 positioned at 90 degrees counterclockwise (relative to the top of the page). The radiation pattern 800, indicated via arrows 816, 818, 820, 822 illustrates how some of the radiation from lamps 808, 810, 812, 814 directed towards mirror surface 804 may be reflected back in predominantly the opposite direction, approximately doubling the radiation in area/direction 824. I should be appreciated that more complex mirrored surfaces may be used, such as focus the UV radiation in a more specific way and/or concentrate the UV radiation on a smaller area. For example, a mirrored surface having multiple ridges may be used, such that the UV radiation may be directed in various different ways. In other examples, different shapes of mirrors may be used at one or more different locations, to tune the exact UV light radiation pattern desired. It should be appreciated that various other modifications are contemplated herein, including selecting one or more of different sizes, shapes, textures, etc., of mirrors (in 2 or 3 dimensions), for a group of UV lamps or specific to individual UV lamps, to achieve the desired reflection pattern of the UV radiation to ultimately increase efficiency of disinfecting various spaces.

FIG. 9 illustrates an example radiation pattern 900 of a mobile disinfecting device 902 with a curved mirror surface 904 positioned at 90 degrees counterclockwise (relative to the top of the page). As illustrated in FIG. 9, the curved mirrored surface 904 may increase the radiation on the opposite side of the mobile disinfecting device 902 by creating a beam of radiation 906 that can be pointed in the desired direction of radiation, represented by arrows 908.

FIGS. 10-12 illustrate different views 1000, 1100, and 1200 of an example room to be disinfected with corresponding heat maps that indicates a degree to which each area in the room is exposed to disinfecting UV light. In some cases, the room may include a hospital room or other area in a hospital, a hotel room, or any room in any space in which it is desirable to disinfect. As illustrated in FIG. 10, room 1002 is defined by walls 1004, 1006, 1008, and 1010, containing objects such as object B 1016, object C 1018, and object D 31020 (e.g., type 2 object), and wall 1014 (e.g., type 1 object).

In some cases, the mobile disinfecting device covers the unobstructed area of the room (e.g., traverse or travels in a least a portion of the room to expose the room to UV light for disinfecting purposes), while accumulating “dosage scores” at some or every location in the room. As illustrated in FIG. 10, the area B 1016 (a narrow couch, for example) is indicated via first type of shading 1026, indicating that object it is above the required threshold dosage score. Region or object A 1012 (e.g., a bathtub), object C 1018 (e.g., a large table), and object D 1020 (e.g., a king-size bed) all have high dosage scores near their edges, but low scores at distances away from the robot coverage area (e.g., in the interior area of the respective objects), as indicated via areas of shading 1022 and 1024. The mobile disinfecting device can return to locations near the low dosage score areas and dwell for a longer time, as the total dosage score accumulates over time, such that all (or predominantly all, such as 90, 95% etc.) of the room is brought to the required threshold dosage levels. The wall 1014 may be identified as a type 2 object, where UV light does not pass through, above or under. Therefore, the dosage score is not recorded for these areas, as indicated by a lack of shading in the area corresponding to the location of wall 1014.

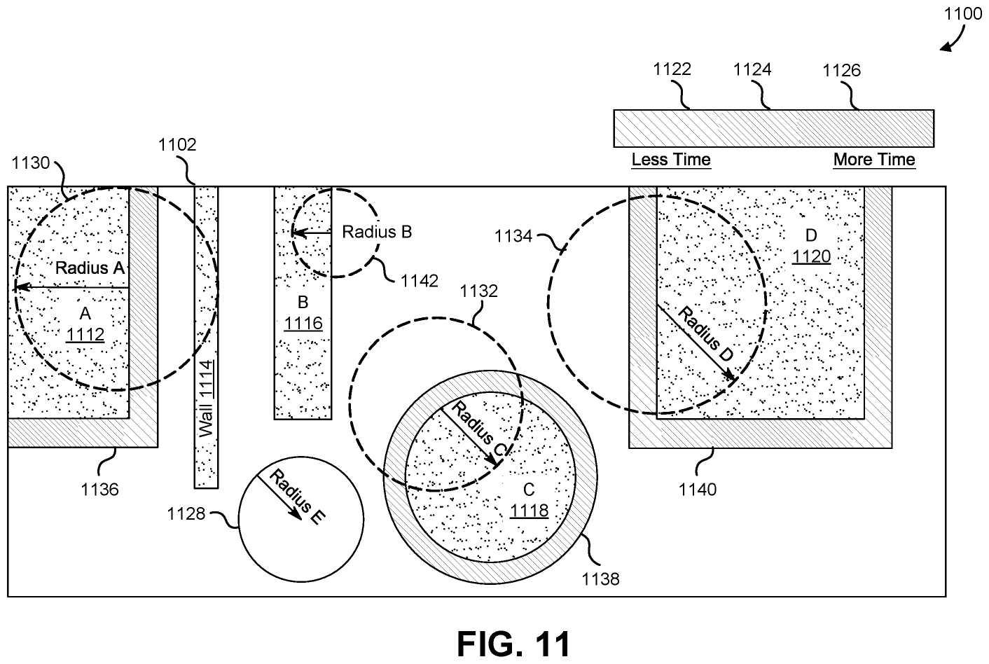

In order to achieve the required/desired dosage for each area of the room, the mobile disinfecting device may need to dwell for a longer time in key areas. An example of a Dwell Time Map 1100 is illustrated by FIG. 11, where the darker areas of the map 1100 of room 1102 (which may be the same room and include the same objects as room 1002 described above in reference to FIG. 10), indicated by shading 1126 in this example may require more time to disinfect the areas further from the mobile UV device. Areas indicated by shading 1124 and 1122 may require less disinfection time. The areas labelled A 1112, B 1116, C 1118, D 1120, and wall 1114 represent obstacles the mobile disinfecting device cannot pass through (e.g., tables, chairs, bed, bathtub, etc.).

The dwell time map 1100 illustrated via shading 1122, 1124, 1116 indicated where the mobile disinfecting device may need to dwell for a longer time as a function of Disinfection Radius or distance from the lamp(s) of the mobile disinfecting device to the location that is being disinfected. In the example illustrated, radius E 1128 may represent the effective radius of the UV lamps, such that within the area defined by radius E from the mobile UV device, full intensity of the UV lamp or lamps are delivered. Objects with dimensions that are less than half the radius E may, such as object B 1116 with a width of radius B 1142, not require added dwell time by the mobile UV device. Objects that do have at least one dimension that is greater than twice the radius E 1128 (or the area around small or larger objects is obstructed by walls or other objects which may be classified as type 1 or type 2 objects thus increasing the accessible radius of these objects to be greater than twice the radius E 1128), may require added dwell time, greater UV lamp intensity, and/or directional UV lamp intensity via operation of one or more mirror devices. These objects may include object A 1112 having a radius A 1130, object C 1118 having a radius C 1132, and object D 1120 having a radius D 1134. In these cases, the mobile disinfecting device may spend longer time (and/or one or more of adjust lamp intensity or direction of one or more lamps), around the accessible perimeter around these objects to yield the desired UV radiation exposure, as indicated by permitters 1136, 1138, 1140 having different areas of shading 1122, 1124, 1126 indicating dwell time. It should be appreciated that as used herein dwell time is correlated to speed/velocity of the mobile UV device, such that a greater dwell time may correspond to a slower speed/velocity and a shorter dwell time may correspond to a faster speed/velocity.

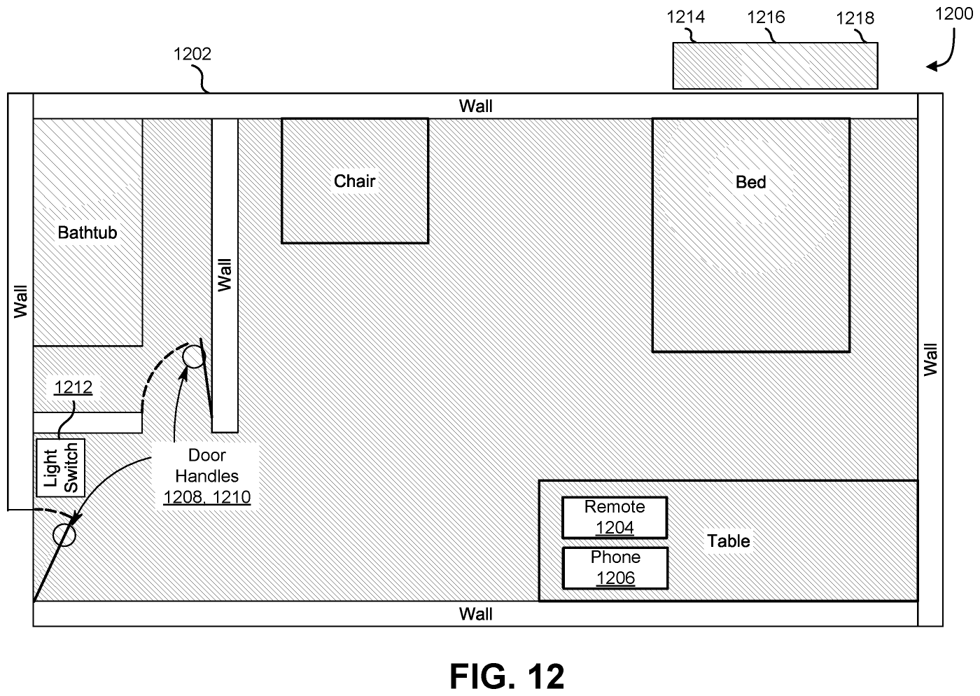

In a similar example illustrated in FIG. 12, the mobile disinfecting device may be programmed to recognize one or more high touch objects, such as one or more of a phone 1204, remote control 1206, door handles 1208, 1210, a light switch 1212, and or other objects or items that may be identified as benefiting form a higher degree of disinfection, in a room 1202 to be disinfected (which may include one or more similar aspects and/or objects as room 1002 described above in reference to FIG. 10). The mobile disinfecting device may need to spend extra time near these objects (as well as other objects described above in reference to FIG. 11), to bring their dosage above their respective threshold levels. As illustrated, shading 1214 may indicate a high dosage score, shading 1216 may indicate moderate dosage score, and shading 1218 may indicate the desired threshold dosage score.

In some cases, the described processes may generate and/or output a heat map, such as any of heat maps 1000, 1100, and/or 1200 for a variety of purposes. In some cases, heat maps 1000 and/or 1200 may be output, with corresponding labels, including, for example, type 1 and type 2 objects, special interest or high touch objects, etc., for various purposes, including to enable operators and/or other systems to verify compliance and/or traceability (e.g., for hospitals or hotels).

In some aspects, the described techniques and systems may be utilized to disinfect entire volumes of space, including, in the enclosed space example, floor, walls, and ceiling, and anything in between. In some instances, disinfection in a certain height band (e.g., ground level to 5, 6, 7 feet or various other dimensions) may be desirable, such as to reduce energy consumption of the device/time to disinfect the space, while still providing the requisite disinfection level to prevent the spread of disease, provide a certain level of sterilization, and so on. In some examples, one or more mirrored surfaced may be paced on the mobile disinfecting device to modify the UV radiation pattern in the vertical dimension as well, such as reach the ground level, or various heights above the top of the one or more UV lamps.

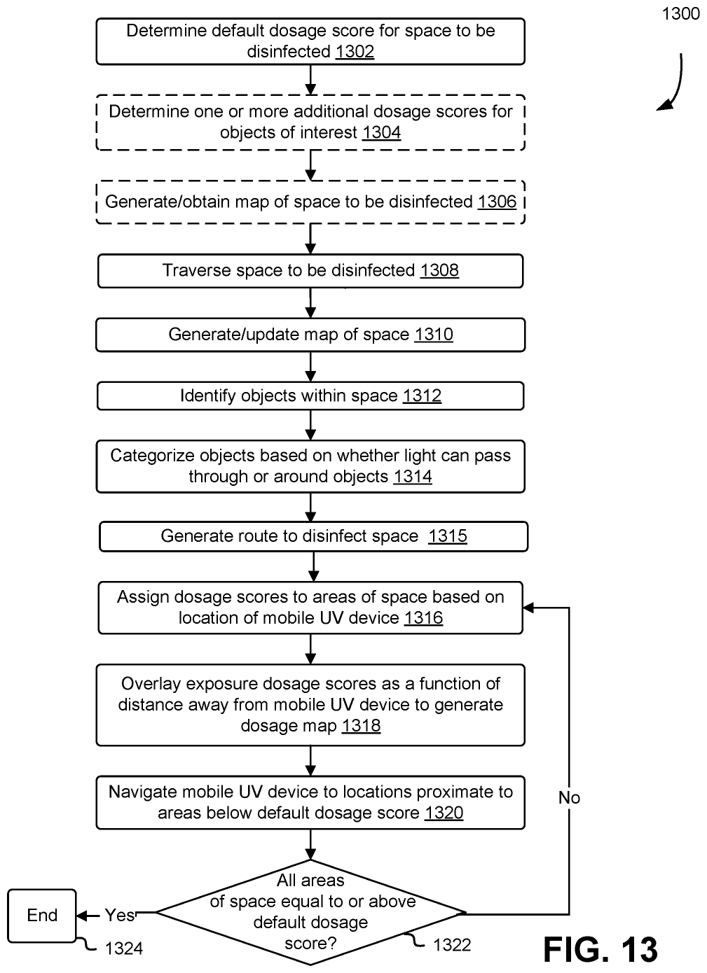

FIG. 13 illustrate an example process 1300 for disinfecting a room, such as may be performed by the mobile disinfecting device 400 and/or 500, described above in reference to FIGS. 4 and 5. In some cases, one or more operations of process 1300 may generate and/or utilize one or more of heat maps 1000, 1100, an/or 1200 described above in reference to FIGS. 10-12. In some cases, process 1300 may include identifying and categories objects, such as into categories, as described above n reference to FIG. 2. It should be appreciated that process 1300 may be utilized to disinfect any space, including internal rooms, hallways, common areas, etc., outdoor spaces, partially enclosed spaces or rooms, and so on. As illustrated in FIG. 13, dotted or dashed lines may indicate optional operations, such that process 1300 may be performed with or without the so indicated operations.

In some aspects, process 1300 may begin at operation 1302, in which a default dosage score for a space to be disinfected (such as an interior room) may be determined or obtained. In some cases, the default dosage score may be set or determined individually for each space to be disinfected, may be set for a type of room to be disinfected (e.g., a relative score of 1000 for a hospital room versus a score of 700 for a hotel room), or may be set based on other characteristics or inputs.

In some optional cases, process 1300 may additionally include operation 1304, in which one or more additional dosage scores may be determined for one or more objects of interest. In some cases, this may include setting one or more additional dosage scores for various classes of objects, such as one score for high touch objects (door knobs, light switches, remote control devices for TV's, etc.), one score may be determined for personal items, such as phones, etc., and another score may be determined for other categories of objects, such as medical devices, personal hygiene devices (e.g., a toothbrush). In other cases, multiple of these different groups of objects may have only a single dosage score. In yet some cases, the default dosage score for the room may be set for these objects.

In some optional implementations, process 1300 may additionally include operation 1306, in which a map of the space to be disinfected may be obtained such as from an external source, or may be generated using various simultaneous localization and mapping (SLAM) techniques. SLAM may include one or more methods, as are known in the art, for an autonomous vehicle, such as the described mobile disinfecting device, that constructs a map of an area based on the vehicle traversing the area, whereby the vehicle may be localized in the map during the generation process. SLAM algorithms enable a vehicle, such as described mobile disinfecting, to map out an unknown environment. In some cases, the mobile disinfecting device may traverse a room or space to be disinfected with or without activating any UV lamps, to map out the room or space and identify different objects within the space, such as to generate a more efficient route or optimized to meet the default dosage score for all or substantially all areas of the space, while reducing or minimizing the time needed to reach the default dosage score. In this example, the mobile disinfecting device may then take a second pass through the space to be disinfected, with a route already mapped out, such as including a path to traverse, whereby different segments of the path may be associated with one or more of a speed (or velocity), a UV lamp intensity or UV radiation pattern characteristic, which may also include a number of lamps powered on or activated, an amount of power supplied to one or more of the UV lamps, a direction of the one or more lamps (e.g., modified via a mirror system), etc., as will be described in greater detail below. In some case, the UV lamp(s) may be powered on for the initial pass at operation 1306, and the route determined later may be take into account the dosage score already associated with the portions of the space that the device has traversed in this first pass.

In some examples, operation 1306 may include obtaining an already generated map of an area to be disinfected, such as may include a floor plan of an interior room, or space, etc., from an external source (e.g., an owner or operator of the space to be disinfected, public records or other records of the space to be disinfected, such as building records, etc.). In this example, the mobile disinfecting device may forego taking a first pass through the space to be disinfected for mapping purposes, as the obtained map may be used instead (such as to aid in generated a more efficient route). In yet other cases, the mobile disinfecting device may still traverse the room a first time to verify accuracy of the map and/or determine if other objects are present, etc.

Process 1300 may proceed, after at least one of operations 1302, 1304, and/or 1306, to operation 1308, in which the mobile disinfecting device may traverse the space to be disinfected, such as with the UV lamp or lamps activated. As the mobile devices moves through the space, a map of the space may be generated and/or updated, at operation 1310, such as via using one or more SLAM techniques. Using various sensors, such as a camera, lidar, sonar, cliff detector, proximity sensor, etc., objects may be identified within the space, at operation 1312. In some optional cases, one or more objects of interest may be identified, such as via one or more sensors of the mobile UV device, at operation 1312. The identified objects may then be categorized based on whether light can pass at least partially through or around the objects, at operation 1314, such as via one or more of the techniques described above in reference to FIG. 2. In some cases, operation 1314 may not utilize specific object recognition techniques, but may rely on the less resource intensive techniques. In some cases, one or both of operations 1312 and 1314 may include generating a point cloud based on whether detected objects are solid or not (e.g., allow light to at least partially pass through them). In some cases, this may include identifying one or more objects via inputs from one or more camera devices and using any of a variety of AI or machine learning techniques to ensure the identification of such objects is above a confidence threshold. Process 1300 may additionally include operation 1315, in which a route may be determined for the mobile disinfecting device to traverse the space to disinfect the entire or substantially all of the space, such as based on the map of the space and the categorized objects within the space.

Dosage scores may be assigned to areas of the space based on the location of the mobile disinfecting device at operation 1316. In some cases, intensity of the UV lamp or lamps, direction or use of mirrors proximate to the UV lamp or lamps, and/or direction of the radiation of the lamps may be taken into account when assigning dosage scores. The dosage scores may then be overlayed onto the map of the space, including overlaying dosage scores for different categories of objects based on or as a function of distance away from the mobile UV device, such as via the techniques described above in reference to FIGS. 10-12, at operation 1318.

Using the dosage map generated/updated at operation 1318, the mobile disinfecting device (or alternatively a backend system in communication with the mobile disinfecting unit) may identify areas that are below the default dosage score (or objects below the specific object dosage score), and navigate or direct the mobile disinfecting device to navigate to or proximate to those areas to increase the dosage score of those areas. Process 1300 may then proceed to operation 1322, in which it may be determined if all of the default/objects of interest dosage scores have been met for all areas of the space. If the determination is negative, process 1300 may loop back through operations 1316, 1318, 1320, and 1322, until the entire room/objects have met or exceeded the default or threshold score, at which point process 1300 may end at operation 1324.

In some cases, operation 1322 may additionally include obtaining and/or using external map data to verify that the mobile disinfecting unit disinfected the entire space (e.g., didn't miss a room, or a closet/bathroom, which may be behind a door, or half wall etc. In some cases, a closed door may be categorized as a type 1 object, and the presence of a detected door knob, hinge, or other feature of the door (e.g., detected using various object detection techniques, machine learning, AI, etc.) may be used to confirm the presence of a door. In this example, external map data, and/or statical information of the room, may be also be used to verify if an area on the other side of the door needs to be disinfected. For example, the mobile disinfecting device may be used to disinfect a large number of hospital rooms, which may share the same dimensions, layout etc. In this example, prior maps of similar rooms at the same address or location, may be used to determine that say a bathroom exists behind a close door that needs to be disinfected. In this example, the room may be flagged, such that some notation is provided with a heat map that is generated and provided in a report of the disinfection of a given room. In some cases, the generated heat map may include square footage or area of the room of space disinfected, such as may be used by an operator to identify if, for instance, a bathroom was not disinfected in a given room based on a closed door, and the like. In other cases, map data may be used in a similar process in place of or in addition to the statistical/historical information of maps generated by the disinfecting devices or a fleet of disinfecting devices (e.g., this or another machine has disinfected the exact same room or space prior).

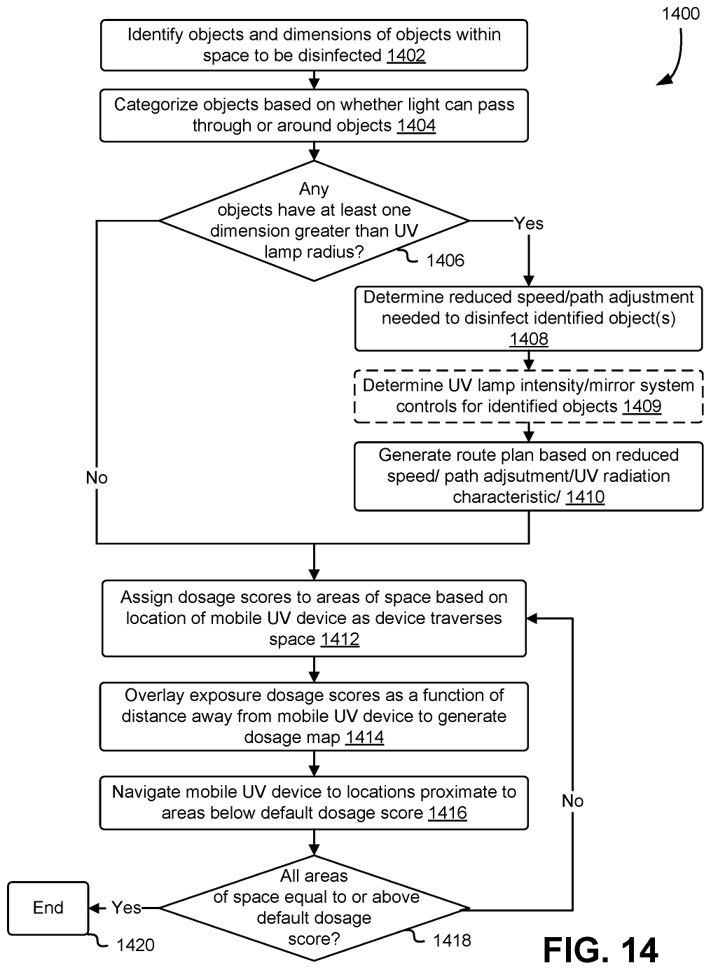

FIG. 14 illustrates another example process 1400 for disinfecting a room, such as may be performed by the mobile disinfecting device 400 and/or 500, described above in reference to FIGS. 4 and 5. In some cases, one or more operations of process 1400 may generate and/or utilize one or more of heat maps 1000, 1100, an/or 1200 described above in reference to FIGS. 10-12. In some cases, process 1400 may include identifying and categories objects, such as into categories, as described above in reference to FIG. 2. It should be appreciated that process 1400 may be utilized to disinfect any space, including internal rooms, hallways, common areas, etc., outdoor spaces, partially enclosed spaces or rooms, and so on. As illustrated in FIG. 14, dotted or dashed lines may indicate optional operations, such that process 1400 may be performed with or without the so indicated operations. In some case, process 1400 may include additional include operations 1302-1310 of process 1300 described above.

Process 1400 may begin at operation 1402, in which one or more objects may be identified within the space to be disinfected, such as via point cloud techniques, which are known in the art. In some optional cases, operation 1402 may also include identifying or determining dimensions of one or more of the objects. In some optional cases, operation 1402 may additionally or alternatively include identifying one or more objects of interest, such as high touch objects and/or highly sensitive objectives, such as with the use of various machine learning and/or object recognition techniques. Operation 1402 may include identifying one or more objects via inputs from one or more camera devices and using any of a variety of AI or object recognition techniques to ensure the identification of such objects is above a confidence threshold. The identified objects may then be categorized based on whether light can pass at least partially through or around the objects, at operation 1404, such as via one or more of the techniques described above in reference to FIG. 2.

In some examples, process 1400 may additionally include operation 1406, in which it may be determined if any of the identified objects (which at least partially allow light to pass through or over them), have at least one dimension that is greater than an ultraviolet radius of the at least one ultraviolet lamp attached to the mobile disinfecting device, such as depicted and described above in reference to FIG. 12. If this determination is positive, then at least one of a speed of the device (e.g., recued speed/increased dwell time), a path of the device. In some cases, one or more characteristics of the space proximate to the identified object under examination may be taken into account, to determine if it may be more efficient (time or energy-wise) for the mobile disinfecting device to travel to another are proximate to the object to enable to provide the require dosage score to the entire object. In some optional cases, process 1400 may additionally or alternatively (e.g., in place of operation 1408), include operation 1409, in which, a UV radiation characteristic (e.g., UV lamp intensity or number of UV lamps active or mirror system control) may be modified to ensure that the entire identified object receives the requisite dosage score. At operation 1410, a route plan may be generated or modified based on the at least one of the reduced speed, modified path, or modified UV radiation characteristic(s) determined at operations 1408 and/or 1409. Process 1400 may then proceed to operation 1412.

For objects that do not have a dimension that is greater than the UV radius, process 1400 may proceed to operation 1412. Dosage scores may be assigned to areas of the space based on the location of the mobile disinfecting device at operation 1412. In some cases, intensity of the UV lamp or lamps, direction or use of mirrors proximate to the UV lamp or lamps, and/or direction of the radiation of the lamps may be taken into account when assigning dosage scores. The dosage scores may then be overlayed onto the map of the space, including overlaying dosage scores for different categories of objects based on or as a function of distance away from the mobile UV device, such as via the techniques described above in reference to FIGS. 10-12, at operation 1412.

Using the dosage map generated/updated at operation 1412, the mobile disinfecting device (or alternatively a backend system in communication with the mobile disinfecting unit) may identify areas that are below the default dosage score (or objects below the specific object dosage score), and navigate or direct the mobile disinfecting device to navigate to or proximate to those areas to increase the dosage score of those areas. Process 1400 may then proceed to operation 1418, in which it may be determined if all of the default/objects of interest dosage scores have been met for all areas of the space. If the determination is negative, process 1400 may loop back through operations 1412-1418, until the entire room/objects have met or exceeded the default or threshold score, at which point process 1400 may end at operation 1420.

As described above, generating and/or modifying a route, which may include speed or dwell time and/or one or more UV radiation characteristics associated with one or more locations or segments of a path through the space to be disinfected, may be an iterative process whereby the route is updated or changed as the mobile disinfecting device moves through the space to be disinfected. In other cases, the route may be determined at a point after a map has been generated (e.g., after an initial traverse of the space), and then modified to the extent that the map was inaccurate. In yet some cases, the heat map may be generated as the mobile disinfecting device traverses the space to be disinfected. In some cases, the heat map may be used to provide for real time updates (e.g., modified instructions to the device) to chance the route to ensure that the threshold dosage score is accumulated across the entire space, such as by an automated system or by an operator monitoring progress of the device. In some cases, the described techniques may be implemented to reduce a time taken to disinfect a space while also conserving power output of the mobile defecting device.

In some aspects, as described herein, a mobile disinfecting device may include an autonomous vehicle unit that includes a propulsion system, at least one of a camera device or a lidar device attached to the autonomous vehicle unit, at least one ultraviolet lamp, attached to the autonomous vehicle unit, usable to disinfect areas proximate to the at least one ultraviolet lamp, at least one processor and memory that stores computer-executable instructions that, as a result of being executed by the one or more processors, cause the mobile disinfecting device to perform the following operations. The instructs may cause the mobile disinfecting device (or system in the case that some of the instructions are executed remotely from the mobile disinfecting device) to generate, using simultaneous localization and mapping, a map of an interior room via traversing at least a portion of the interior room by the autonomous vehicle unit. Next, using at least one input from at least one of the camera device or the lidar device, a plurality of objects in the interior room may be identified. Each of the plurality of objects may be categorized as a first type object or a second type object to generate a set of categorized objects based on the at least one input from at least one of the camera device or the lidar device, where the first type object comprises a first object that does not permit light to pass through or around the first object, and n the second type object comprises a second object that at least partially permits light to pass through or around the second object.

Next, a route for the mobile disinfecting device to travel to disinfect the interior room may be determined based on the map and the set of categorized objects, where the route includes a path and speed of the mobile disinfecting device corresponding to different segments of the path to yield a dosage score that at least meets or exceeds a minimum disinfection threshold value for the interior room. In some cases, the dosage score is based on ultraviolet exposure from the at least one ultraviolet lamp to different portions of the interior room. In some cases, determining the route may additionally include determining at least one of a reduced speed, a modified path, or an ultraviolet radiation characteristic for at least one segment of the different segments of the path corresponding to the mobile disinfecting unit moving proximate to at least one second type object, to satisfy or exceed the disinfection threshold value for the at least one second type object.

In some cases, at least one of the plurality of objects may be categorized as a second type object based additionally on at least one dimension of the second type object exceeding a radiation radius of the at least one ultraviolet lamp, such as described above in reference to FIG. 11. In some aspects, a heat map may be generated based on the map of the interior room, where the heat map includes indications of a dosage score for different portions of the interior room, such as described above in reference to FIGS. 10 and 12. In some cases, the mobile disinfecting device may also include a mirror system proximate to the at least one ultraviolet lamp, where the mirror system includes at least one movable mirror surface for changing the ultraviolet radiation characteristic of the at least one ultraviolet lamp. In this example, at least a portion of radiation may be redirected from the at least one ultraviolet lamp towards the at least one second type object by causing the at least one mirror surface to interrupt at least a portion of the radiation pattern of the at least one ultraviolet lamp, such as to increase the dosage score for that type 2 object, without requiring the mobile disinfecting device to slow down as much to reach the desired dosage score.

In some cases, determining at least the ultraviolet radiation characteristic may include changing at least one of an intensity of the at least one ultraviolet lamp or a number of the at least one ultraviolet lamps upon the mobile disinfecting device moving proximate to the at least one second type object. In some aspects, at least one third type object m ay be identified, where the third type object includes at least one of a high touch object or a highly sensitivity object. In this example, at least one of a second reduced speed, a second modified path, or a second ultraviolet radiation characteristic may be determined for the mobile disinfecting device for at least one second segment of the different segments of the path corresponding to the mobile disinfecting unit moving proximate to at least one third type object, to satisfy or exceed the disinfection threshold value for the at least one third type object.

In some aspects, it may be determined that at least one of the identified first type objects corresponds to a shadow region relative to the mobile disinfecting device. Based on the determined shadow region, at least one of a second reduced speed, a second modified path, or a second ultraviolet radiation characteristic for the mobile disinfecting device may be determined for at least one second segment of the different segments of the path corresponding to the mobile disinfecting unit moving proximate to the shadow region, to satisfy or exceed the disinfection threshold value for the shadow region.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives. Additionally, elements of a given embodiment should not be construed to be applicable to only that example embodiment and therefore elements of one example embodiment can be applicable to other embodiments. Additionally, in some embodiments, elements that are specifically shown in some embodiments can be explicitly absent from further embodiments. Accordingly, the recitation of an element being present in one example should be construed to support some embodiments where such an element is explicitly absent.

What is claimed is:

1. A mobile disinfecting device comprising:
an autonomous vehicle unit comprising a propulsion system;
at least one of a camera device or a lidar device attached to the autonomous vehicle unit;
at least one ultraviolet lamp, attached to the autonomous vehicle unit, usable to disinfect areas proximate to the at least one ultraviolet lamp;
at least one processor; and
memory that stores computer-executable instructions that, as a result of being executed by the one or more processors, cause the mobile disinfecting device to:
generate, using simultaneous localization and mapping, a map of an interior room via traversing at least a portion of the interior room by the autonomous vehicle unit;
identify, using at least one input from at least one of the camera device or the lidar device, a plurality of objects in the interior room;
categorize each of the plurality of objects as a first type object or a second type object to generate a set of categorized objects based on the at least one input from at least one of the camera device or the lidar device, wherein the first type object comprises a first object that does not permit light to pass through or around the first object, and wherein the second type object comprises a second object that at least partially permits light to pass through or around the second object; and
determine a route for the mobile disinfecting device to travel to disinfect the interior room based on the map and the set of categorized objects, the route comprising a path and speed of the mobile disinfecting device corresponding to different segments of the path to yield a dosage score that at least meets or exceeds a minimum disinfection threshold value for the interior room, the dosage score based on ultraviolet exposure from the at least one ultraviolet lamp to different portions of the interior room, wherein determining the route further comprises:
determine at least one of a reduced speed, a modified path, or an ultraviolet radiation characteristic for at least one segment of the different segments of the path corresponding to the mobile disinfecting unit moving proximate to at least one second type object, to satisfy or exceed the disinfection threshold value for the at least one second type object.

2. The mobile disinfecting device of claim 1, wherein the computer-executable instructions further include instructions that further cause the mobile disinfecting device to:
categorize at least one of the plurality of objects as a second type object based additionally on at least one dimension of the second type object exceeding a radiation radius of the at least one ultraviolet lamp.

3. The mobile disinfecting device of claim 1, wherein the computer-executable instructions further include instructions that further cause the mobile disinfecting device to:
generate a heat map based on the map of the interior room, the heat map comprising indications of a dosage score for different portions of the interior room.

4. The mobile disinfecting device of claim 1, further comprising a mirror system proximate to the at least one ultraviolet lamp, the mirror system comprising at least one movable mirror surface for changing the ultraviolet radiation characteristic of the at least one ultraviolet lamp, wherein the computer-executable instructions further include instructions that further cause the mobile disinfecting device to:
redirect at least a portion of radiation from the at least one ultraviolet lamp towards the at least one second type object by causing the at least one mirror surface to interrupt at least a portion of the radiation pattern of the at least one ultraviolet lamp.

5. The mobile disinfecting device of claim 1, wherein determining at least the ultraviolet radiation characteristic further comprises changing at least one of an intensity of the at least one ultraviolet lamp or a number of the at least one ultraviolet lamps upon the mobile disinfecting device moving proximate to the at least one second type object.

6. The mobile disinfecting device of claim 1, wherein the computer-executable instructions further include instructions that further cause the mobile disinfecting device to:
identify at least one third type object, the third type object comprising at least one of a high touch object or a highly sensitivity object, wherein determining the route further comprises:
determine at least one of a second reduced speed, a second modified path, or a second ultraviolet radiation characteristic for the mobile disinfecting device for at least one second segment of the different segments of the path corresponding to the mobile disinfecting unit moving proximate to at least one third type object, to satisfy or exceed the disinfection threshold value for the at least one third type object.

7. The mobile disinfecting device of claim 1, wherein the computer-executable instructions further include instructions that further cause the mobile disinfecting device to:
determine that at least one of the identified first type objects corresponds to a shadow region relative to the mobile disinfecting device; and
based on the determined shadow region, determine at least one of a second reduced speed, a second modified path, or a second ultraviolet radiation characteristic for the mobile disinfecting device for at least one second segment of the different segments of the path corresponding to the mobile disinfecting unit moving proximate to the shadow region, to satisfy or exceed the disinfection threshold value for the shadow region.

8. A computer-implemented method comprising:
identifying, by a mobile disinfecting device comprising at least one ultraviolet lamp and at least one sensor device, using the at least one sensor device, a plurality of objects in a space to be disinfected;
categorizing at least a subset of the plurality of objects as a first type object or a second type object to generate categorized objects based on at least one input from the at least one sensor device, wherein the first type object does not permit light to pass through or around the first object, and wherein the second type object permits light to pass at least partially through or around the second object; and
determining a route for the mobile disinfecting device to travel to disinfect the space based on the map and the categorized objects, the route comprising a path and speed of the mobile disinfecting device corresponding to different segments of the path to yield a dosage score that at least meets or exceeds a minimum disinfection threshold value for different areas of the space, the dosage score based on ultraviolet exposure from the at least one ultraviolet lamp to different portions of the interior room, wherein determining the route further comprises:
determining at least one of a reduced speed, a modified path, or an ultraviolet radiation characteristic for at least one segment of the different segments of the path corresponding to the mobile disinfecting device moving proximate to at least one second type object, to satisfy or exceed the disinfection threshold value for the at least one second type object.

9. The computer-implemented method of claim 8, further comprising:
generating a heat map based on the map of the space, the heat map comprising indications of a level of cumulative ultraviolet exposure to the different portions of the interior room.

10. The computer-implemented method of claim 8, wherein determining the route for the mobile disinfecting device to travel to disinfect the space further comprises determining or modifying at least of the path or the speed concurrently with the mobile disinfecting device traveling on the route.

11. The computer-implemented method of claim 8, wherein the minimum disinfection threshold value for the space comprises a cumulative amount of ultraviolet exposure at a plurality of locations equally disposed within the space, and wherein determining the route further comprises:
determining at least one of an ultraviolet radiation intensity or an ultraviolet radiation pattern corresponding to different segments of the path to yield at least the minimum disinfection threshold value for different areas of the space.

12. The computer-implemented method of claim 11, wherein determining at least one of the ultraviolet radiation intensity or the ultraviolet radiation pattern comprising causing at least one mirrored surface proximate to the at least one ultraviolet lamp to be placed at least partially block a portion of the ultraviolet radiation pattern.

13. The computer-implemented method of claim 8, further comprising:
determining that at least one of the identified first type objects corresponds to a shadow region relative to the mobile disinfecting device; and
based on the determined shadow region, determining at least one of a second reduced speed or a second modified path for the mobile disinfecting device for at least one second segment of the different segments of the path corresponding to the mobile disinfecting unit moving proximate to the shadow region, to satisfy or exceed the disinfection threshold value for the shadow region.

14. The computer-implemented method of claim 8, further comprising:
identifying at least one third type object, the third type object comprising at least one of a high touch object or a highly sensitivity object, wherein determining the route further comprises:
determining at least one of a second reduced speed or a second modified path for the mobile disinfecting device for at least one second segment of the different segments of the path corresponding to the mobile disinfecting unit moving proximate to at least one third type object, to satisfy or exceed the disinfection threshold value for the at least one third type object.

15. The computer-implemented method of claim 8, further comprising:
based on detecting at least one moving object with the at least one sensor device, at least one of: modifying the route or modifying operation of the at least one ultraviolet lamp to avoid directing ultraviolet radiation towards the moving object.

16. The computer-implemented method of claim 8, wherein the at least one sensor device comprises at least one of a camera device, a lidar device, a sonar device, a proximity sensor, or a cliff sensor.

17. A non-transitory computer-readable storage medium storing thereon executable instructions that, as a result of being executed by one or more processors of a computer system, cause the computer system to at least:
identify, via inputs from at least one sensor device, a plurality of objects in a space to be disinfected;
categorize each of the plurality of objects as a first type of object or a second type of object to generate categorized objects based on at least one input from at least one of the camera device or the lidar device, wherein the second type of object comprises a second object that permits light to pass through or around the second object; and
determine a route for a mobile disinfecting device comprising at least one ultraviolet lamp, to travel to disinfect the space based on the map and the categorized objects, the route comprising a path and speed of the mobile disinfecting device corresponding to different segments of the path to yield a dosage score that at least meets or exceeds a minimum disinfection threshold value for different areas of the space, the dosage score based on ultraviolet exposure from the at least one ultraviolet lamp to different portions of the space, wherein determining the route further comprises:
determine at least one of a reduced speed or an ultraviolet lamp intensity for the mobile disinfecting device for at least one segment of the different segments of the path corresponding to the mobile disinfecting device moving proximate to at least one second type object, to satisfy or exceed the disinfection threshold value for the at least one second type object.

18. The non-transitory computer-readable storage medium of claim 17, wherein the instructions further comprise instructions that, as a result of being executed by the one or more processors, cause the computer system to:
determine that at least one of the identified first type objects corresponds to a shadow region relative to the mobile disinfecting device; and
based on the determined shadow region, determine at least one of a second reduced speed or a second ultraviolet lamp intensity for the mobile disinfecting device for at least one second segment of the different segments of the path corresponding to the mobile disinfecting unit moving proximate to the shadow region, to satisfy or exceed the disinfection threshold value for the shadow region.

19. The non-transitory computer-readable storage medium of claim 17, wherein the instructions further comprise instructions that, as a result of being executed by the one or more processors, cause the computer system to:
identify at least one third type object, the third type object comprising at least one of a high touch object or a highly sensitivity object, wherein determining the route further comprises:
determine at least one of a second reduced speed or a second ultraviolet lamp intensity for the mobile disinfecting device for at least one second segment of the different segments of the path corresponding to the mobile disinfecting unit moving proximate to at least one third type object, to satisfy or exceed the disinfection threshold value for the at least one third type object.

20. The non-transitory computer-readable storage medium of claim 17, wherein the instructions further comprise instructions that, as a result of being executed by the one or more processors, cause the computer system to:
modify the ultraviolet lamp intensity by causing at least one mirrored surface proximate to the at least one ultraviolet lamp to be placed at least partially block a portion of the ultraviolet radiation pattern.
